(19) United States
(12) Patent Application Publication
(10) Pub. No.: US 2011/0104239 A1
(43) Pub. Date: May 5, 2011

(54) PROBIOTIC CHEWING GUM METHOD OF MANUFACTURE

(75) Inventors: Holly A. Knutsen, Palos Park, IL (US); Pamela A. Mazurek, Orland Park, IL (US); Narendra K.B. Shah, Bolingbrook, IL (US); Daniel J. Sitler, Naperville, IL (US); Claire A. Theis, Chicago, IL (US)

(73) Assignee: WM. WRIGLEY JR. COMPANY, Chicago, IL (US)

(21) Appl. No.: 13/000,505

(22) PCT Filed: Jun. 24, 2009
(86) PCT No.: PCT/US2009/048433

§ 371 Date: Dec. 21, 2010

Related U.S. Application Data
(60) Provisional application No. 61/075,182, filed on Jun. 24, 2008.

(51) Int. Cl. A61K 9/68; A61P 1/00

(52) U.S. Cl. 424/440; 424/48

(57) ABSTRACT

An extruded, centerfilled or coated confectionary material is provided which contains a probiotic which is capable of having a shelf life greater than about six months. A chewing gum production method is also provided which produces stick, centerfilled and coated chewing gum with probiotics without substantial loss of probiotics due to excess water, heat or pressure. Probiotics may be included in the chewing gum mass of the stick or coated chewing gum, in the liquid or powder center of a centerfilled gum, in the coating of the coated chewing gum, or as an additional layer upon the stick. The probiotics delivered to the oral cavity provide an oral health benefit to the consumer through suppression of pathogenic bacteria.

### BACKGROUND OF THE INVENTION

This invention relates to manufacture of a confectionery material such as chewing gum containing a probiotic and more particularly relates to a process and apparatus for manufacture of a probiotic-containing chewing gum.

Consumers recognize chewing gum as providing oral care benefits and as a delivery mechanism for oral health ingredients. In addition to mechanical cleaning of the teeth provided by the chewing action, saliva stimulated by chewing, flavor and taste from the product conveys beneficial properties in reducing bad breath, neutralizing acid, and remineralizing teeth. Saliva also contains beneficial polypeptides and other components which may improve the oral environment. Representative polypeptides include antimicrobial proteins, such as lysozyme, lactoferrin, peroxidases, and histatins and inhibitors of spontaneous crystallization, such as statherin.

Chewing gum is recognized as being a delivery mechanism for oral health ingredients. Typical oral health ingredients include whitening agents such as sodium bicarbonate, remineralization ingredients agents such as calcium carbonate and calcium lactate, and antibacterial agents such as magnolia bark extract. These oral health ingredients are not metabolic in nature and therefore do not have the processing and shelf life complications associated with sustaining the viability of a biologically active agent such as a probiotic.

The chewing gum of the present invention includes a biologically active agent. Specifically, biologically active agents included in the chewing gum are probiotics.

There is considerable and growing interest in products containing probiotics, that is, microorganisms which confer a health benefit on a host. Health benefits include improved digestive function, improved immunity to disease, and improved oral health.

An example of a product containing probiotics is yogurt. Probiotics typically are found as live, active organisms in yogurt. Active probiotics are retained in a yogurt product over time by maintaining the probiotics in an aqueous-based medium containing adequate nutrients, and typically slowing metabolism of the probiotics by maintaining the yogurt in a cooled environment such as through refrigeration. Thus, a yogurt product may have a reasonable shelf life before and after purchase by a consumer. However, a product such as a chewing gum does not have an aqueous-based medium in which active probiotics may be maintained. Further, chewing gum products normally are not cooled before or after purchase by a consumer but are stored under ambient conditions.

Probiotics may be placed in an inactive or dormant state, which are capable of being reactivated. Probiotics typically are made inactive through a process such as freeze drying which removes moisture from live, active probiotics under low temperature and low pressure. In many instances, an inactive probiotic may be reactivated through contact with water. Thus, inactive probiotics can confer a health benefit to a host once reactivated through hydration such as in the oral cavity or in another location of the digestive tract of the host.

Once inactive, probiotics should be handled and processed carefully to avoid being killed or becoming ineffective through environmental or mechanical conditions such as excess heat, pressure, or shear. The term “ineffective” as used herein means a state in which probiotics cannot confer a clinically measured health benefit to the host even after exposure to conditions which result in probiotic activity. Inactive probiotics must also be handled and processed to avoid being prematurely hydrated and reactivated prior to consumption. If activated prematurely, probiotics will metabolize available nutrients and will die or become ineffective.

During processing, inactive probiotics typically should avoid high shear mixing, high temperatures, high pressures, high moisture and other conditions which may kill or make ineffective the probiotics. A consumer product containing inactive probiotics must resist conditions such as high moisture which could prematurely activate the probiotic during the shelf life of the product.

A consumer product, such as a chewing gum, involves substantial challenges for inclusion of an inactive probiotic capable of being reactivated. Chewing gum, commercially distributed as pieces (which further may be coated or filled), typically is produced by combining chewing gum components including a gum base, flavors, sweeteners, fillers, and binders in a mixer; extruding such combined components into a slab; rolling such slab into a uniform flat sheet of a desired thickness and width; scoring the uniform flat sheet into individual sticks; and ultimately packaging the resulting sticks.

Traditional chewing gum manufacturing processes and methods pose unique challenges for maintaining probiotics in an inactive state. For example, a confectionary product such as a chewing gum which has been manufactured as a pressed tablet may expose the inactive probiotics to high pressure, compression or forces which may kill probiotics, although probiotics may survive in pressed tablets, especially if the composition of the tablet is non-homogeneous. Additionally, chewing gum mixed and later extruded may expose the probiotics to high sheer and high temperatures, which may be fatal to probiotics. Finally, chewing gum which is later coated using traditional coatings may expose the probiotics during a coating process to excess moisture which may activate the probiotics.

Additionally, chewing gum storage and shelf life requirements pose unique challenges. Generally, chewing gum may be stored for six to twelve months. During this time, the chewing gum may be exposed to moisture which activates the probiotics. If the probiotics activate during storage, the probiotics will die or become ineffective prior to being consumed by the consumer.

Preferably, probiotics delivered from a chewing gum in an inactive state are intended to be released from the product during mastication (chewing). Upon hydration in the oral cavity, probiotics released into the mouth, reactivate, and attach within the oral cavity and to the oral mucosa. Activated probiotics confer a health benefit to the consumer and may help to suppress pathogenic bacteria, such as Streptococcus mutans associated with dental caries and the volatile-sulfur forming bacteria (“VSC bacteria”) that contribute to halitosis. Thus, there is a need for a chewing gum product which contains inactive probiotics which are activatable during chewing and thereby produce a health benefit.

### SUMMARY OF THE INVENTION

Accordingly, the present invention is directed to a confectionary composition containing an inactive probiotic, which is activatable upon contact with water and which is capable of having a probiotic effectiveness shelf life greater than about six months. More particularly, the present invention is directed to an extruded, centerfilled or coated chewing gum containing an inactive probiotic, which is activatable upon contact with water and which is capable of having a probiotic effectiveness shelf life greater than six months.

The present invention is further directed to a process to produce a stick, centerfilled or coated chewing gum containing a probiotic. The process comprises: (a) forming a chewing gum mass, (b) incorporating at least one probiotic into the chewing gum mass to form a probiotic chewing gum, and (c) converting the probiotic chewing gum into a stick, tablet, centerfilled or coated chewing gum, wherein the probiotics are not made ineffective during the process.

The present invention is still further directed to a process for producing a chewing gum containing probiotics. The process comprises: (a) mixing chewing gum components to form a chewing gum having a surface and a core; and (b) applying inactive probiotics to the surface of the chewing gum, wherein the probiotics are not substantially activated due to rehydration.

The present invention is still further directed to a process for delivering probiotics to the oral cavity for providing an oral health benefit. The process comprises: (a) delivering a stick or coated chewing gum containing inactive probiotics into the oral cavity; and (b) activating the inactive probiotics through masticating the chewing gum.

The present invention is still further directed to a process for producing a two-layered chewing gum. The process comprises: (a) compressing a first chewing gum formulation to form a first layer, wherein the amount of compression is less than about 10 kN, and (b) compressing a second chewing gum formulation to form a second layer, wherein the amount of compression is less than about 30 kN.

### BRIEF DESCRIPTION OF THE DRAWING

FIG. 1 is a schematic view of a manufacturing process according to embodiments of this invention.

FIG. 2 is a cross-sectional view of a coated chewing gum.

FIG. 3 is a cross-sectional view of chewing gum having a surface application.

FIG. 4 is a cross-sectional view of chewing gum having a liquid center.

### DESCRIPTION OF THE INVENTION

In one embodiment of the method of this invention, an extruded, centerfilled or coated confectionery product, such as a chewing gum, is produced containing inactive probiotics, which may be reactivated during consumption or use. Typically, a chewing gum of this invention containing such an inactive probiotic may be maintained under conditions in which water content is kept below a level which causes reactivation of the probiotic.

A chewing gum may be manufactured with probiotics through a sheeting (extrusion) process, a centerfilled extrusion process or a tableting process. The sheeted or tableted probiotic chewing gum may be coated with a coating which may contain additional probiotics. Additionally or alternatively, a chewing gum may be manufactured without probiotics and then have probiotics applied with a layer or coating which includes probiotics. Accordingly, embodiments of methods of the present invention may involve preparation of a confectionary material that includes a probiotic within the confectionary material or coating the material.

The confectionery material may be any hard candy, soft candy, chewing gum, or other confectionery substance, or compound that has a fluid phase or may take a flowable form. In other words, the confectionery material may be any material that may be heated, melted, form a syrup, or be dissolved in a liquid to become flowable as is commonly known in the art. Nonlimiting examples of suitable confectionery materials that are flowable or may be placed into a flowable state include syrups, liquids or solids for making hard candies, soft candies, lollipops, fondants, toffees, jellies, chewing gums, chocolates, gelatins and nougats. The confectionery material may include sugar or may be sugar-free. Coloring may be added to the confectionery substrate as desired.

The preferred confectionery produced according to this invention is a chewing gum. Typically, a chewing gum material suitable for use in the process of this invention is composed of probiotics, water-insoluble gum base, flavorings, sweeteners, high intensity sweeteners (HIS), bulking agents such as polyols, and may contain other components such as fillers and binders. Typically during mastication, the probiotic releases with a portion of the flavor and water soluble ingredients over a period of time. The gum base portion is retained in the mouth throughout the chew.

The term “probiotic” refers to microorganisms, generally bacteria, which when administered in adequate amounts confer a health benefit on the host. Health benefits may include those relating to gut health, oral health, and immune health. Microorganisms generally recognized as probiotics are Bacillus coagulans, Bacillus subtilis, Bacillus laterosporus, Bacillus laevolacticus, Sporolactobacillus inulinus, Lactobacillus acidophilus, Lactobacillus curvatus, Lactobacillus reuteri, Lactobacillus jenseni, Lactobacillus casei, Lactobacillus fermentum, Lactobacillus rhamnosus, Lactobacillus johnsonii, Lactobacillus salvalarius, Lactococcus lactis, Pedioccocus acidilacti, Pedioccocus pentosaceus, Pedioccocus urinae, Leuconostoc mesenteroides, Bacillus coagulans, Bacillus subtilis, Bacillus laterosporus, Bacillus laevolacticus, Sporolactobacillus inulinus, Bifidobaterium bifidum, Bifidobaterium animalis, Bifidobaterium breve, Bifidobaterium infantis, Bifidobaterium lactis, Bifidobaterium longum and mixtures thereof.

Probiotics utilized in confectionery products of embodiments of the present disclosure may be selected from a species of the group consisting of Bacillus, Lactobacillus, Pedioccocus, Leuconostoc, Bifidobaterium and mixtures thereof. Probiotics may also be selected from the group consisting of Lactobacillus acidophilus, Lactobacillus reuteri, Lactobacillus plantarum, Lactobacillus salvalarius, Lactobacillus sporogenes, Bifidobaterium longum and mixtures thereof. One microorganism specifically identified as a probiotic is Lactobacillus plantarum 299v. Lactobacillus plantarum 299v is commercially available from Probi AB, Ideon Gamma 1, Sölvegatan 41, Lund, Sweden.

One of the constituents of chewing gums of embodiments of the present invention is gum base. The term “gum base” refers to a composition containing elastomers, elastomer solvents, plasticizers, waxes, emulsifiers, and/or inorganic fillers. Plastic polymers, such as polyvinyl acetate, which behave somewhat as plasticizers, may also be included in the gum base. Other plastic polymers that may be used include polyvinyl laurate, polyvinyl alcohol, and polyvinyl pyrrolidone. Gum base typically comprises about 20 to about 40% of the overall chewing gum composition. (All composition percents herein are in percent by weight unless otherwise stated.) However, in less common formulations, the overall chewing gum composition may comprise as low as about 5% or as high as about 95% gum base. The gum base may also include a filler component. The filler component may be an inorganic powder such as calcium carbonate, magnesium carbonate, talc, dicalcium phosphate, tricalcium phosphate or the like. The filler may constitute from about 5 to about 50% of the gum base. Occasionally, a portion of the filler may be added to the chewing gum mixture separately from the gum base.

Chewing gums of the present disclosure may be flavored (i.e., include one or more flavors). The term “flavor” refers to an ingredient employed to impart a characteristic aroma and taste sensation to chewing gum products. Most flavors are water insoluble liquids but water soluble liquids and solids are also known. These flavors may be natural or artificial in origin. Flavors are typically employed at levels of about 0.1 to about 4% of the finished gum product. It is common to co-dry and encapsulate flavors with various carriers and/or diluents. For example, spray-dried flavors using gum Arabic, starch, cyclodextrin or other carriers are often used in chewing gum for protection, controlled release, control of product texture and easier handling as well as other reasons. When flavors are in such forms, it will often be necessary to increase the usage level to compensate for the presence of the carriers or diluents. In the event that flavors are known to have an antibacterial effect or other effect upon probiotics, flavors may be spray-dried or encapsulated to prevent interaction with the probiotics. Alternatively, probiotics may be delivered through non-aqueous flavor carriers.

Chewing gums of the present disclosure may include high intensity sweeteners (HIS). In the case of sugarless gums, it is usually desirable to add high intensity sweeteners to compensate for the reduced sweetness resulting from substitution of sugar alcohols for the sucrose in sugar gums. More recently, the trend has been to also add high intensity sweeteners to sugar gums to boost and extend flavor and sweetness. High intensity sweeteners (which are sometimes called high potency or artificial sweeteners) may be defined as food acceptable chemicals which are at least twenty times sweeter than sucrose. Commonly used high intensity sweeteners include aspartame, sucralose, and acesulfame-K. Less common are saccharin, thaumatin, alitame, neotame, cyclamate, perilla derived sweeteners, stevia derived sweeteners, monatin, monellin and chalcones. Usage levels for high intensity sweeteners may vary widely depending on the potency of the sweetener, local market preferences and the nature and level of other ingredients which might impart bitterness to the gum. Typical levels can range from about 0.01 to about 2%, although some applications may dictate usage outside that range. These sweeteners may be combined together, or with non-high intensity sweeteners at varying levels to impart a sweetness synergy to the overall composition.

Chewing gums of the present disclosure may include bulking agents. The majority of the water soluble portion of the chewing gum will typically comprise a water-soluble, powdered carbohydrate which serves as a bulking agent. In sugar gums, this most often is sucrose although other sugars such as fructose, erythrose, dextrose (glucose), levulose, tagatose, galactose, trehalose, corn syrup solids and the like, alone or in any combination may also be used. Generally, sugarless chewing gums will employ sugar alcohols (also called alditols, polyols or polyhydric alcohols) as bulking agents due to their benefits of low cariogenicity, reduced caloric content and reduced glycemic values. Such sugar alcohols include sorbitol, mannitol, xylitol, hydrogenated isomaltulose (isomalt), maltitol, erythritol, hydrogenated starch hydrolysate solids, and the like, alone or in any combination. Longer chain saccharides such as polydextrose and fructo-oligosaccharides are sometimes employed for their reduced caloric properties and other health benefits. The bulking agents typically comprise about 5 to about 95% of the gum composition.

Water (moisture) may be added as a separate ingredient but is more often a minor component of other added ingredients. While almost all food ingredients contain some water, most of the water is contributed by carbohydrate syrups where present. Other components which may contribute significant amounts of moisture include certain bulking agents, glycerin and occasionally other ingredients. The total amount of moisture in a chewing gum product is important to texture and stability of the probiotics. If not sufficiently protected by packaging, chewing gums may gain or lose moisture to the surrounding environment. In the confectionary materials and chewing gums of this invention, water content may be maintained at concentrations at which significant amounts of inactive probiotics are unable to reactivate. Thus, such materials, especially chewing gums, are capable of maintaining a commercially-reasonable shelf life under normal storage conditions. Moisture levels in chewing gums containing probiotics may be as little as about 0.1% or even less. Probiotic chewing gum should contain minimal amounts of moisture, preferably in the range of about 0 to about 1% but may be as high as about 3% depending on the presence of moisture sensitive ingredients and other factors. In certain embodiments, the chewing gum may contain from about 0.5 to about 2.5% water or from about 0.5 to about 1% water. Typically, moisture levels greater than about 1% may activate probiotics and greater than about 3% may activate probiotics to an extent to render the probiotics ineffective prior to a commercially-reasonable shelf life.

Confectionary materials and chewing gums of embodiments of this invention are capable of maintaining a commercially-reasonable shelf life under normal storage conditions. A shelf life is at least about six months, preferably at least about nine months and more preferably is at least about twelve months under typical conditions of temperature and humidity. Shelf life may be determined by testing a product after a period of time to determine whether the product retains normal characteristics such as flavor, consistency, softness, and the like. Shelf life for an included probiotic may be tested for effectiveness under “real time” conditions or 23° C. and 50% relative humidity. Shelf life may be determined by accelerated techniques using elevated temperature and humidity or 35° C. and 85% relative humidity.

A “probiotic effectiveness shelf life” according to the present disclosure is the length of time at which the product maintains probiotic activity above a threshold necessary to confer a health benefit to the consumer at a given serving size. A threshold for an oral health benefit is typically greater than about 1×10⁶ CFU, 1×10⁷ CFU, 1×10⁸ CFU in a total serving size or serving per day. Probiotics for an oral health benefit in the amount of 1×10⁸ CFU a total serving size or serving per day may be delivered in one piece at about 1×10⁸ CFU per piece, two pieces at about 5.0×10⁷ CFU per piece, four pieces at about 2.5×10⁷ CFU per piece, or six pieces or about 1.67×10⁷ CFU per piece. A threshold for a gut health benefit is typically greater than about 1×10⁷ CFU, 1×10⁸ CFU or 1×10⁹ CFU in a total serving size or serving per day. Probiotics for a gut health benefit in the amount of 1×10⁹ CFU a total serving size or serving per day may be delivered in one piece at about 1×10⁹ CFU per piece, two pieces at about 5.0×10⁸ CFU per piece, four pieces at about 2.5×10⁸ CFU per piece, or six pieces or about 1.67×10⁸ CFU per piece.

Piece size, measured in weight, containing probiotics may be between about 0.5 to about 5.0 grams, preferably between about 1.0 to about 2.0 grams, and more preferably between about 1.3 to about 1.5 grams.

Process conditions during manufacture of a product according to this invention should be controlled as to avoid conditions which would render a probiotic ineffective. Thus, excessive (i.e. conditions at which effectiveness of a probiotic is adversely affected) heat, cold, pressure, or shear should be avoided.

In an embodiment of a process of this invention, a probiotic may be added to a confectionary material or chewing gum during various stages of manufacture. Preferably, if a manufacturing process requires excessive conditions of temperature, pressure, or shear, a probiotic is added after such process conditions are completed.

Preferably, a product of this invention containing an activatable inactive probiotic is capable of being stored under typical storage conditions for a commercially-reasonable time. Further, such a product does not normally require water-tight packaging such as a wholly foil sealed package (such as a foil blister package). Typical packages for confections or gum useful for this invention may be a bottle or have a plastic wrapping which is gas permeable, but not water tight. Shelf life typically is determined for products contained in such packages. For example, an isolated stick of gum may be fresh for only a few weeks outside a plastic wrapped package, but will have a shelf life of about six to about twelve months in such a package. Shelf life for products of this invention are determined typically in normal consumer packaging such as with plastic wrapping, but not sealed in foil.

In some embodiments, the probiotic chewing gum specifically provides an oral health benefit to the consumer. The term “oral health benefit” refers to a benefit conferred to the host's oral cavity, (i.e., inside of the mouth including surfaces of the teeth, gums, tongue, and other tissue in the mouth). Examples of oral health benefits include a reduction in the number of volatile sulfur compound (VSC) forming bacteria and S. mutans in the oral cavity, preferably a reduction of about 20% of the total VSC forming bacteria, pathogenic bacteria and S. mutans in the oral cavity, and more preferably a reduction of about 50% of the total number of colony forming units of VSC forming bacteria and S. mutans in the oral cavity. Other oral health benefits include reduction of dental caries, reduction of gum inflammation, and reduction of off-odors typical of “bad-breath” and halitosis. The probiotics chewing gum may function to reduce the number of VSC forming bacteria, pathogenic bacteria and S. mutans in the oral cavity by removing the bacteria from the oral cavity and/or by suppressing (i.e., decreasing the growth rate of undesirable bacteria).

In an embodiment, the chewing gum is produced, extruded and scored laterally and longitudinally into sticks. As used herein, “sticks” include common product forms produced upon an extruder including traditional sticks such as Wrigley's Extra® as marketed in the United States and tabs such as Wrigley's Orbit® as marketed in the United States. As herein defined, sticks also include other products which may be produced upon an extruder such as chunk style bubble gum, bubble tape, and liquid filled.

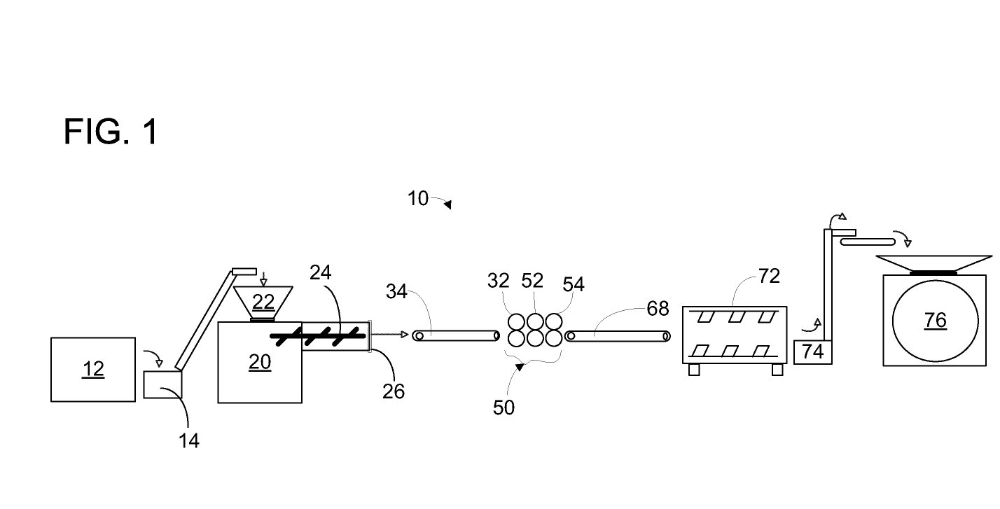

FIG. 1 illustrates a stick forming apparatus of the present invention. Apparatus 10 includes a continuous or batch mixer 12 which forms a chewing gum mass from ingredients. A typical throughput of apparatus 10 is between about 500 and about 5500 kg/hour (about 1100 and about 12,000 lbs/hour), more preferably between about 2750 and about 4500 kg/hr (about 6000 and about 10,000 lbs/hour), and sized to at least accommodate the maximum throughput of apparatus 10. Chewing gum mixed in the mixer 12 has a typical output temperature between about 50 and about 53° C. (about 122 to about 127° F.). In some embodiments, the chewing gum formed in the mixer has a temperature of less than about 55° C. (about 131° F.) or even from about 40 to about 50° C. (about 104 to about 122° F.). The probiotics may be incorporated at higher temperatures (such as above about 55° C. (about 131° F.)); however, if incorporated at such higher temperatures, it is preferred that the mixture be rapidly cooled below about 55° C.

Probiotics may be added to the mixer 12 directly as an ingredient or using a pre-blend containing probiotics and one or more polyols. A pre-blend is useful to evenly distribute the probiotics in the chewing gum mass more quickly as compared to probiotics added directly to the chewing gum mass. The pre-blend may include polyols in liquid or powder form. The pre-blend may also contain powdered ingredients such as magnesium stearate and other powder ingredients which are resistant to moisture and talc and other filler powders which are not reactive with probiotics or an acid produced by the probiotics which is present during processing and storage of the chewing gum (i.e., lactic acid).

Limiting time in the mixer 12 is essential as the probiotics are heat and/or shear sensitive and exposure to heat and/or shear time should be limited to prevent the probiotics from being killed or become ineffective. Mixer residence time is typically limited to between about 15 and about 30 minutes, preferably less than about 20 minutes, more preferably less than about 15 minutes and still more preferably less than about 10 minutes. It has been found that probiotics may be incorporated using a standard batch mixing process but added to the mixer at the end of mixing or less than the last 7 minutes of batch mixing, preferably less than the last 5 minutes of batch mixing, and more preferably less than the last 2 minutes of batch mixing. Such mix times should function to distribute the probiotics throughout the chewing gum mass but limit the probiotics exposure to heat and/or shear time.

The mixer 12 discharges chewing gum to a conveyor 14. The chewing gum may be in the form of a chewing gum loaf, a continuous extrudate, a semi-continuous extrudate or other forms or strands of chewing gum composition.

The chewing gum mass enters a forming extruder 20 via a receiving hopper 22. An extruder screw 24 or extruder screws receive chewing gum from the hopper 22. Probiotics may be added to the extruder screw 24 directly as an ingredient or using a pre-blend containing probiotics and one or more polyols to avoid exposure to heat and/or shear associated with the mixer 12.

The extruder screw 24 extrudes the chewing gum through die 26 into a slab. The extruder screw 24 may include a water jacket with water circulating at between typically about 48 and about 50° C. (about 118 to about 122° F.). Additionally, the extruder 20 adds heat to the chewing gum by friction. The temperature of the composition in the extruder 20 is such to permit movement through the extruder but not at such a high temperature as to kill the probiotics or render them ineffective. Typically the temperature of the composition exiting the extruder 20 is less than about 53° C. (about 127° F.), preferably less than about 50° C. (about 122° F.) and still preferably less than about 49° C. (about 120° F.). The temperature of the composition exiting the extruder 20 should not be a temperature which prevents the composition from becoming overly viscous and typically is above about 37° C. (about 98° F.), preferably above about 43° C. (about 110° F.) and more preferably above about 47° C. (about 116° F.). To prevent a substantial loss of viability of the probiotics in the chewing gum, the temperature of the composition exiting the extruder 20 should not exceed about 55° C. (about 131° F.).

A slab of chewing gum may be in a continuous ribbon or regular slab format. Typically, ribbon thickness is about 3 to about 12 cm (about 1 to about 5 in) and more preferably between about 5 and about 10 cm (about 2 and about 4 in). A regular slab may have a width between preferably about 10 and about 76 cm (about 4 and about 30 in) and more typically about 45 to about 60 cm (about 18 and about 24 in). Alternatively, chewing gum may be extruded from die 26 in a thin slab. The thin slab may have a thickness typically from about 0.15 to about 3 cm (about 0.06 to about 1.2 in), and more typically, from approximately 0.4 cm (about 0.16 in) to approximately 0.5 cm (about 0.20 in). This slab may have a typical width from approximately 10 cm (about 4 in) to approximately 70 cm (about 28 in) wide and more typically from about 2 to about 55 cm (about 1 to about 22 in) wide. The thickness of the slab may depend upon whether the chewing gum is to be formed into a chewing gum stick, tab, or pellet. The thickness of the chewing gum slab helps reduce the time the chewing gum can be cooled to a level which prevents the probiotics from being killed or become ineffective.

The chewing gum exiting the die 26 may be pressed through one or more calender rolls (not shown), which size slab or ribbon exiting the die or smooth out any surface irregularities. The chewing gum is transferred from the die 26 to a conveyor 34. The conveyor 34 and an environmental closure (not shown) which may be designed to rapidly cool the chewing gum to prevent the probiotics from being killed or become ineffective.

A forming unit 50 receives the chewing gum slab from the conveyor 34 and sizes the slab into its final stick format. The forming unit 50 may include calender rolls 32, cross scoring unit 52 and a circular scoring unit 54. Scoring rollers may be chilled to further reduce the temperature of the chewing gum slab and prevent the probiotics from being killed or become ineffective.

Upon exiting the forming unit 50, the chewing gum sticks may enter a post-scoring conveyer 68 with an environmental closure (not shown) which may be designed to rapidly cool and control relative humidity of the chewing gum sticks.

The sticks exiting the scoring conveyor 68 may be stored in a tempering room at a relatively constant temperature and humidity to avoid the probiotics from being killed or becoming ineffective. The sticks are later packaged or may be further processed through coating by passing into a breaking device 72 to form individual pieces and through conveyor 74 to a coater 76.

The apparatus 10 may be a dedicated production line for the production of probiotic chewing gum. Alternatively, the apparatus may be used for both probiotic chewing gum and chewing gum that does not contain probiotics. Prior to running the apparatus for a chewing gum that does not contain probiotics, the apparatus 10 is sanitized. The sanitation process includes running a cleaning batch through the entire line starting at the mixing process and ending at the scoring process. The cleaning batch consists of gum base and an absorbent polyol such as sorbitol. Antimicrobial flavors such as peppermint oil or menthol can also be added to the cleaning batch. Alternatively, food approved sanitation chemicals may be utilized including Alconox® to sanitize certain parts of the gum processing line.

As an alternative of the manufacturing process of FIG. 1, a chewing gum mass may be used which is suitable for tableting and preservation of the probiotics. Polyols suitable for the chewing gum mass are selected to minimize moisture uptake and chewing gum base is selected which does not react to an acid produced by the probiotics (i.e., lactic acid).

Probiotics may then be added to the chewing gum mass and tableted. The probiotics should be tableted using a process which limits the probiotics exposure to excess heat and/or compression force which may kill or render the probiotics ineffective. Compression forces less than about 30 kN are preferred, less than about 25 kN is more preferred and less than about 20 kN with a variability of +/−about 5 kN is still more preferred. Additionally, precompression forces should be minimized and are preferably less than about 10 kN, more preferably less than about 8 kN and still more preferably between about 0 and about 5 kN.

In some embodiments, a two-layered compressed chewing gum may be produced. Typically, a two-layered product is produced by subjecting a first layer to a precompression force and a compression force and then adding a second layer which is together with the first layer are subjected to a precompression force and a compression force. Generally, the two-layered compressed chewing gum containing probiotics may be produced limiting exposure to compression forces by introducing the formulation of the first layer (which may be the same or different from the formulation of the second layer) into the press and applying a compression force. Generally, the force applied to form the first layer may be less than about 20 kN, less than about 10 kN or even less than about 5 kN. In some embodiments, the pressure applied is from about 1 kN to about 10 kN. After the first layer is pressed, the formulation of the second layer is loaded into the press with the product (e.g., tablet) of the first compression, and then compression is applied once again. The force applied to the second layer may be less than about 50 kN, less than about 40 kN or even less than about 30 kN. In one embodiment, the force is from about 15 kN to about 35 kN. Probiotics may be present in the first layer and/or the second layer. As described more fully in Example 4, it is believed that by applying compressive forces as described, less probiotics are destroyed during the chewing gum manufacturing process.

In another embodiment, in a first stage, a center pressed tablet is made which includes probiotics and in a second stage undergoes a separate tableting process to enrobe the center pressed tablet.

The tableted chewing gum may be coated to inhibit water migration to the gum mass. Similar coating techniques are useful for both extruded chewing gum illustrated in FIG. 1 and compressed chewing gum. The preferred coating method utilizes an open pan coater which utilizes a stainless steel pan with drying air supplied from outside the pan. Alternatively, the coating mechanism may be a perforated pan which permits air flow into the pan through perforated holes in the circumference of the pan. The perforated pan has a larger capacity than the open pan coater; however, the open pan coater is more easily sanitized.

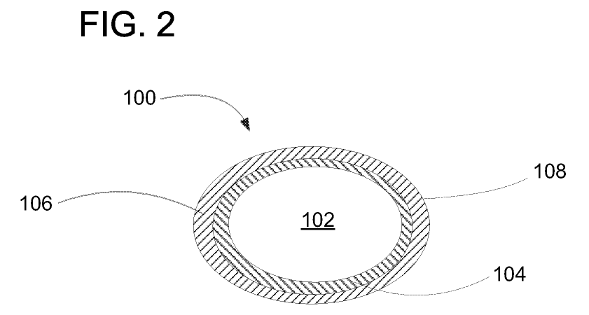

If probiotics are included in a chewing gum core, a precoat may be added to prevent excess moisture from coming in contact with the gum core and activating the probiotics. One such precoat is a mixture of gum talha, magnesium stearate, and flow agents such as silicon dioxide, titanium dioxide, and aluminum oxide. This precoat has been found to create a barrier to the gum core and prevent the probiotics from being activated during a standard coating process in which multiple layers of alternating aqueous syrup and dry charge additions are added to the coating. Other precoating barriers include wax, shellac and other moisture resistant barriers. FIG. 2 illustrates a probiotic coated chewing gum 100 in which the gum core 102 includes probiotics. A barrier layer 104 is utilized to prevent an aqueous carrier of the coating 106 from contacting the gum core 102 to such an extent that the probiotics are killed or rendered ineffective. Finally, a layer of wax polish or shellac 108 is added to give the outer surface of the chewing gum pellet a shiny appearance.

To produce the chewing gum, a dry pre-coat may be first made by mixing a composition containing about 66.6% gum tahla (a gum glue which helps keeps the coating on the surface of the gum) and about 33.3% of a water repelling substance such as magnesium stearate. The chewing gum pellets are tumbled and dry charged for approximately 20 minutes so that powder adheres to the pellet surface and a dry pre-coat is formed on the surface of the uncoated chewing gum. After the dry pre-coat is applied, then several applications of a water based syrup coating containing either saturated sucrose or a polyol solution are added. The syrup coating phase includes adding several coating layers through a process involving: adding a syrup application, adding a dry charge application to accelerate coating build up, a pause or distribution phase, and a drying phase. Finally, a layer of wax polish or shellac is added.

In an experiment, probiotics were incorporated into chewing gum pellet and the actual probiotic concentration was measured to be 1.0×10⁷ cfu/g. The chewing gum pellet was coated with the dry pre-coat described above and the probiotic concentration was measure to be 2.8×10⁶ cfu/g. Thus, the coated chewing gum pellet had a 28% probiotic viability.

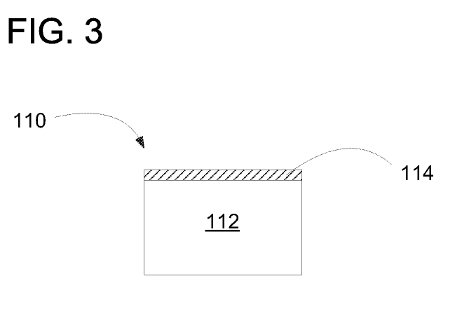

In an alternative method of incorporating probiotics into the chewing gum, a spraying system (not shown) may be positioned upon the conveyer 34 (FIG. 1), the post-scoring conveyor 68 or offline (not shown) to apply probiotics to the surface of the chewing gum slab or chewing gum pieces. A non-aqueous carrier is preferred to prevent the probiotics from becoming activated. Representative non-aqueous carriers include propylene glycol, non-aqueous flavors and dies, and lipids, or wax-based carriers. The probiotics are applied to the surface at pressure and temperature which do not kill the probiotics or render the probiotics ineffective. FIG. 3 illustrates a cross section of product 110 which includes a chewing gum layer 112 and a layer of probiotics 114. Colors and flavors may be added to the non-aqueous carrier.

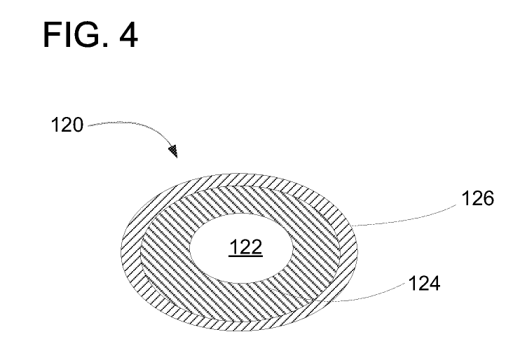

In another method of incorporating probiotics into chewing gum and as seen in FIG. 4, a centerfilled chewing gum 120 may be produced wherein probiotics are at the center of the chewing gum. The centerfilling 122 of the chewing gum may be a powder or a liquid. If the centerfilling is a powder, the probiotics may be added to the center as a powder with a preblend of bulking agents such as polyols and flow agents. If the centerfilling is a liquid, the probiotics may be added as a suspension to a liquid which is non-aqueous or contains a low level of water. The probiotics in the center may compliment probiotics in either or both a gum layer 124 and a coating layer 126.

Alternatively or in addition, probiotics may be included in a bead that is incorporated into the chewing gum. Beads are often in the form of gelatin beads which have an outer gelatin shell with a liquid non-aqueous center containing probiotics. Beads may also have an outer shell made of other materials, such as alginate or alginate polyol blends. The gelatin beads are added to a chewing gum mass and protect the probiotics during processing and shelf-life.

### EXAMPLES

### Example 1

### Determination of Probiotic Survival in Extruded Gums Containing Various Bulking Agents and with Various Packaging

Seven gum compositions were prepared by mixing the various components at a temperature of about 55° C. The components of the gum and the percent inclusion of each component are shown in Table 1 below. The probiotic pre-blend included a 9 to 1 mass ratio of sorbitol to lactobacillus plantarum 299V.

TABLE 1
Percent Inclusion by Weight of Various Components
of the Gums Tested in Example 1

| Component (%) | Run 1 | Run 2 | Run 3 | Run 4 | Run 5 | Run 6 | Run 7 |
|---|---|---|---|---|---|---|---|
| Alpha Sorbitol | 70.09 | 0 | 0 | 0 | 0 | 0 | 0 |
| Erithritol | 0 | 70.09 | 0 | 0 | 0 | 0 | 0 |
| Isomalt | 0 | 0 | 70.09 | 0 | 0 | 0 | 0 |
| Maltitol | 0 | 0 | 0 | 70.09 | 0 | 0 | 0 |
| Mannitol | 0 | 0 | 0 | 0 | 70.09 | 0 | 0 |
| Xylitol | 0 | 0 | 0 | 0 | 0 | 70.09 | 70.09 |
| Gum Base - | 26 | 26 | 26 | 26 | 26 | 0 | 26 |
| Calcium Carbonate | | | | | | | |
| Gum Base - Talc | 0 | 0 | 0 | 0 | 0 | 26 | 0 |
| Probiotic Pre-blend | 0.83 | 0.83 | 0.83 | 0.83 | 0.83 | 0.83 | 0.83 |
| Encapsulated | 0.7 | 0.7 | 0.7 | 0.7 | 0.7 | 0.7 | 0.7 |
| Acesulfame | | | | | | | |
| potassium | | | | | | | |
| Acesulfame | 0.03 | 0.03 | 0.03 | 0.03 | 0.03 | 0.03 | 0.03 |
| potassium | | | | | | | |
| (unencapsulated) | | | | | | | |
| Encapsulated | 0.5 | 0.5 | 0.5 | 0.5 | 0.5 | 0.5 | 0.5 |
| Aspartame | | | | | | | |
| Spearmint Oil | 1.85 | 1.85 | 1.85 | 1.85 | 1.85 | 1.85 | 1.85 |

The various components were added to the mixer after the period of time shown in Table 2 with “0” being the start of mixing. Mixing was completed after 18 minutes.

TABLE 2
Temporal Point of addition of Gum Components

| Component | Time |
|---|---|
| Bulking Agent | 6 |
| Gum Base | 0 |
| Probiotic Pre-blend | 16 |
| High Intensity Sweeteners | 14 |
| Spearmint Oil | 7 |

A portion of probiotic gum from Run 1 was placed in a bottle that included a desiccant and a portion was placed in a foil bag. This was also done for gum from Run 6 and Run 7. The theoretical concentration of probiotic organisms in each of the gums before packaging was 4.98×10⁸ cfu/g. The probiotic concentration after 1 month, 3 months and 7 months is shown in Table 3. The actual survival of the probiotic organism after extrusion and 1 month after storage ranged from 3.8×10⁸ cfu/g to 3×10⁷ cfu/g with an average of 1.9×10⁸ cfu/g.

TABLE 3
Probiotic concentrations after storage for 1, 3 and 7 months

| | | 1 Month | 3 Month | 7 Month |
|---|---|---|---|---|
| | | Concentration | Concentration | Concentration |
| Sample | Packaging | (cfu/g) | (cfu/g) | (cfu/g) |
| Run 1 | Bottle with | 3.8 × 10⁸ | 4.6 × 10⁷ | 9.1 × 10⁶ |
| | Desiccant | | | |
| Run 1 | Foil Bag | 4.3 × 10⁷ | 7.0 × 10⁵ | 2.6 × 10⁷ |
| Run 6 | Bottle with | 3.7 × 10⁸ | 2.5 × 10⁸ | 3.7 × 10⁶ |
| | Desiccant | | | |
| Run 6 | Foil Bag | 6.3 × 10⁷ | 2.0 × 10⁶ | <1 × 10⁵ |
| Run 7 | Bottle with | 2.6 × 10⁸ | 5.3 × 10⁷ | <1 × 10⁵ |
| | Desiccant | | | |
| Run 7 | Foil Bag | 3 × 10⁷ | 2.5 × 10⁶ | <1 × 10⁵ |

A weight gain experiment was conducted in a desiccator held at 75% relative humidity. The tests concluded that the chewing gum using sorbitol (Run 1) as a bulking agent had the greatest percentage of moisture gain. Chewing gums with isomalt, erythritol, isomalt, maltitol, mannitol and xylitol were found to have less moisture gain than sorbitol. The formulation of Run 6 using a xylitol polyol and a talc base was chosen for scale up testing.

TABLE 4
Percentage of weight gain from moisture absorption

| Time (days) | 0 | 1 | 2 | 5 | 7 | 9 | 13 | 21 |
|---|---|---|---|---|---|---|---|---|
| Run 1 | 0 | 0.933 | 2.124 | 3.041 | 3.153 | 3.676 | 4.390 | 5.627 |
| Run 2 | 0 | 0.150 | 0.712 | 0.620 | 0.403 | 0.483 | 0.483 | 0.575 |
| Run 3 | 0 | 0.817 | 1.339 | 1.384 | 1.294 | 1.430 | 1.520 | 1.767 |
| Run 4 | 0 | 0.221 | 0.541 | 0.442 | 0.255 | 0.288 | 0.266 | 0.321 |
| Run 5 | 0 | 0.158 | 0.552 | 0.406 | 0.192 | 0.237 | 0.226 | 0.260 |
| Run 6 | 0 | 0.171 | 0.782 | 0.905 | 0.714 | 0.782 | 0.849 | 1.006 |
| Run 7 | 0 | 0.263 | 0.668 | 1.057 | 0.798 | 1.022 | 0.998 | 1.081 |

### Example 2

### Preparation of a Probiotic-Containing Extruded Chewing Gum

Three batches of chewing gum containing probiotics were mixed weighing approximately 485 kg each. Run 1 contained probiotics added to deliver a theoretical initial loading of 5.0×10⁶ CFU/serving. Run 2 contained probiotics added to deliver a theoretical initial loading 5.0×10⁷ CFU/serving. Run 3 contained probiotics added to deliver a theoretical initial loading 1.0×10⁸ CFU/serving.

The three batches included a probiotic pre-blend of alpha sorbitol and a probiotic, lactobacillus plantarum 299V (Probi AB, Lund, Sweden) in a 9:1 ratio. The addition of sorbitol to the probiotic assisted in bulking up the probiotic for addition to the batch mixer and also to dry out the probiotic which had been previously refrigerated.

The batches were mixed by first adding gum base to a batch mixer. Next, bulking agents or polyols were added to the mixer after 6 minutes. After another 1 minute, mint oil was added. After another 7 minutes, artificial sweeteners were added. After another 2 minutes the probiotic pre-blend was added. The mixture was mixed for another 2 minutes to incorporate the probiotics pre-blend. The resulting gum mixture was soft but still suitable for gum production by extrusion. Extrusion was carried out using conventional means and process conditions, such that the extruded gum had a temperature upon exiting the extruder of 47.2° C.

TABLE 5
Percent Inclusion by Weight of Various Components
of the Gums Tested in Example 2

| Component (%) | Run 1 | Run 2 | Run 3 |
|---|---|---|---|
| Xylitol | 62.9 | 62.837 | 62.753 |
| Mannitol | 8 | 8 | 8 |
| Gum Base | 26 | 26 | 26 |
| Probiotic Pre-blend | 0.0083 | 0.083 | 0.167 |
| Encapsulated Acesulfame | 0.7 | 0.7 | 0.7 |
| potassium | | | |
| Acesulfame potassium | 0.03 | 0.03 | 0.03 |
| (unencapsulated) | | | |
| Encapsulated Aspartame | 0.5 | 0.5 | 0.5 |
| Mint Oil | 1.85 | 1.85 | 1.85 |

TABLE 6
Probiotic viable post-processing and % viable post processing

| | | Post- | Post- | Post- | |
|---|---|---|---|---|---|
| | | Processing | Processing | Processing | % Viable |
| | Theoretical | Rep 1 | Rep 2 | Avg | Post |
| Sample | (cfu/g) | (cfu/g) | (cfu/g) | (cfu/g) | Processing |
| Run 1 | 5 × 10⁶ | 5 × 10⁵ | 8 × 10⁵ | 6.5 × 10⁵ | 13% |
| Run 2 | 5 × 10⁷ | 2.9 × 10⁷ | 5.9 × 10⁶ | 1.74 × 10⁷ | 34.8% |
| Run 3 | 1 × 10⁸ | 2.9 × 10⁷ | 2.9 × 10⁷ | 2.65 × 10⁷ | 26.5% |

### Example 3

### Determination of the Effect of Mixing Time on Probiotic Survival

A compressed gum containing probiotics was prepared in two runs and the mixing time was varied for each run. In the first trial, the four different formulations shown in Table 7 were prepared. Portions of each formulation were placed in different types of packaging, including blister packaging, a bottle with a desiccant present in the bottle, a bottle without any desiccant present, and a tube.

TABLE 7
Formulations Produced in the First Trial

| | Formulation 1 | Formulation 2 | Formulation 3 | Formulation 4 |
|---|---|---|---|---|
| Base | All In-Gum - | All In-Gum - | All In-Gum - | All In-Gum - |
| | Sorbitol | Sorbitol | Sorbitol | Sorbitol |
| Probiotics | Probiotic | Probiotic only | Probiotic | Probiotic |
| | Preblend with | (no pre-blend) | Preblend with | Preblend with |
| | xylitol | | xylitol | xylitol |
| Flavor | Liquid Flavor at | Liquid Flavor at | Liquid Flavor at | Liquid Flavor at |
| | 1% by weight | 1% by weight | 1.7% by weight | 0.5% by weight |
| | | | | and spray dried |
| | | | | flavor at 5.22% |
| | | | | by weight |

Preparation of the formulations of Table 7 began by preparation of the probiotic pre-blend. A low shear tumbling mixer (L. B. Bohle D-59320, Type LM40) housed in a humidity controlled room was used for mixing. A 90:10 weight ratio of xylitol to lactobacillus plantarum 299V (Probi AB, Lund, Sweden) was used in the pre-blend. A master batch of liquid flavor, All-In-Gum SF (available from CAFOSA GUM S.A., Calabria 267, 08029 Barcelona Spain) and talc was prepared. In Formulation 4, an additional spray dried flavor was included.

All ingredients were weighed out in humidity controlled environments. Formulation 2 was mixed for 20 minutes and the other three formulations were mixed for 29 minutes. The liquid flavor was atomized into the master blend over a 1 to 2 minute period. In Formulation 2, the probiotics were directly added to the layers of the product and a probiotic pre-blend was not used.

For Formulation 2, the master blend was split into a 40:60 weight ratio. Color, magnesium stearate and the probiotic powder were added to the master blend for the 40% layer in a low shear tumbling mixer. The mixer was allowed to tumble for 10 minutes at 30 rpm. After this layer was finished, it was sieved due to clumping. Magnesium stearate and the probiotic powder where then added to the second portion of the master blend and mixed for 20 minutes.

Based on the amount of probiotics added to Formulations 1-4 for a 1.3 gram piece, each Formulation should have had a probiotic count of about 5.5×10⁸ CFU/g. However, 20 minutes of mixing resulted in a reduction of the count for Formula 1 to 2.89×10⁷ CFU/g or a 5.2% viable; for Formula 2 to 3.12×10⁷ CFU/g or a 5.73% viable; for Formula 3 to 2.43×10⁷ CFU/g or a 4.4% viable; and for Formula 4 to 4.075×10⁷ CFU/g or a 7.4% viable

Each formulation was pressed (Fette 3090 Bi-layered (Tp9B2 and TP9B1)) after the master batch was produced in a humidity controlled environment. During pressing, the temperature was 21.3° C. and the relative humidity was 31%.

For Formulation 2, each individual layer pressed well but the two layers did not stick well together. The pressing pressure was lowered to achieve adequate adhesion. For Formulation 1, a single layered tablet was pressed. The pressing trial was run for 30 minutes. In this time, there were no problems and the formula pressed well. For Formulation 3, pressing started well but target piece size could not be achieved over time. As the pressing continued, material began to stick to the top punch. The machine had to be stopped and cleaned for the next batch. Formulation 4 did not press as well as the other prototypes. From the beginning, the material was sticking to the top punch causing the machine to stop.

All formulas were packaged in 4 packaging types: PVDC blisters, bottles with a desiccant added, bottle without a desiccant and finally a tube that has a built in desiccant in the cap. The product was stored overnight in high barrier bags. The bottles and tubes were manually packed. The blisters were packed on an automated blister line. All four package types were put into stability testing at both accelerated and real time conditions.

The formulations and packaging were tested at two conditions: 35° C. and 85% relative humidity and 23° C. and 50% relative humidity. Tables 8-11 below show the bacteria counts for the different test conditions for Formulations 1 and 2.

TABLE 8
Stability Results for Formulation 1
at 35° C. and 85% relative humidity.

| | | Bottle with | | Bottle w/o |
|---|---|---|---|---|
| Time | Blister | Desiccant | Tube | Desiccant |
| 0 days | 4.30E+07 | 2.80E+07 | 6.60E+06 | 3.80E+07 |
| 1 day | 3.65E+07 | 4.40E+07 | 2.60E+07 | 2.50E+08 |
| 2 days | 2.50E+07 | 4.30E+07 | 2.65E+07 | 6.00E+07 |
| 3 days | 2.80E+07 | 1.71E+07 | 1.75E+07 | 6.85E+07 |
| 5 day | 8.80E+06 | 3.10E+07 | 2.00E+07 | 4.85E+07 |
| 7 days | 3.40E+06 | 5.20E+06 | 5.40E+06 | 7.75E+06 |
| 10 days | 8.00E+06 | 2.75E+07 | 9.50E+06 | 9.50E+06 |
| 14 days | 1.50E+06 | 2.00E+06 | 1.70E+07 | 1.50E+06 |
| 21 days | 1.00E+05 | 4.90E+06 | 1.07E+07 | 3.00E+05 |
| 28 days | <1.0E+05 | 9.00E+06 | 1.40E+07 | <1.0E+05 |

All results reported as CFU/g. The CFU/g average of the T = 0 days is post-processing viability average or 2.89 × 10⁷ CFU/g.

TABLE 9
Stability Results for Formulation 1
at 23° C. and 50% relative humidity.

| | | Bottle with | | Bottle w/o |
|---|---|---|---|---|
| Time | Blister | Desiccant | Tube | Desiccant |
| 0 days | 4.30E+07 | 2.80E+07 | 6.60E+06 | 3.80E+07 |
| 1 month | 3.55E+07 | 2.70E+07 | 3.55E+07 | 3.25E+07 |
| 2 month | 4.70E+06 | 2.80E+07 | 2.65E+07 | 2.17E+07 |
| 3 month | 1.75E+06 | 3.35E+07 | 1.79E+07 | 3.20E+06 |
| 4 month | <1.0E+05 | 1.95E+07 | 1.07E+07 | 3.00E+05 |
| 5 month | <1.0E+05 | 4.10E+07 | 4.60E+06 | <1.0E+05 |
| 6 month | <1.0E+05 | 2.40E+07 | 7.05E+06 | <1.0E+05 |

All results reported as CFU/g. The CFU/g average of the T = 0 days is the post-processing viability average or 2.89 × 10⁷ CFU/g.

TABLE 10
Stability Results for Formulation 2
at 35° C. and 85% relative humidity.

| | | Bottle with | | Bottle w/o |
|---|---|---|---|---|
| Time | Blister | Desiccant | Tube | Desiccant |
| 0 days | 3.30E+07 | 3.10E+07 | 6.90E+06 | 5.40E+07 |
| 1 day | 9.30E+07 | 6.30E+07 | 3.75E+07 | 9.20E+07 |
| 2 days | 4.15E+07 | 6.75E+07 | 2.90E+07 | 5.40E+07 |
| 3 days | 3.70E+07 | 2.85E+07 | 2.65E+07 | 7.80E+07 |
| 5 days | 8.90E+06 | 1.20E+07 | 2.65E+07 | 3.55E+07 |
| 7 days | 7.85E+06 | 6.00E+07 | 2.80E+07 | 5.45E+07 |
| 10 days | <1E+05 | 7.90E+07 | 4.00E+07 | 6.70E+06 |
| 14 days | 2.00E+05 | 3.25E+07 | 2.55E+07 | 1.02E+07 |
| 21 days | <1E+05 | 3.80E+07 | 4.75E+07 | 8.00E+05 |
| 28 days | <1E+05 | 4.90E+07 | 7.50E+06 | 1.00E+05 |

All results reported as CFU/g. The CFU/g average of the T = 0 days is the post-processing viability average or 3.12 × 10⁷ CFU/g.

TABLE 11
Stability Results for Formulation 2
at 23° C. and 50% relative humidly.

| | | Bottle with | | Bottle w/o |
|---|---|---|---|---|
| Time | Blister | Desiccant | Tube | Desiccant |
| 0 days | 3.30E+07 | 3.10E+07 | 6.90E+06 | 5.40E+07 |
| 1 month | 3.35E+07 | 7.25E+07 | 1.85E+07 | 6.00E+07 |
| 2 month | 5.85E+06 | 8.95E+07 | 3.35E+07 | 3.10E+07 |
| 3 month | 1.35E+06 | 5.45E+07 | 2.25E+07 | 3.15E+06 |
| 4 month | <1.0E+05 | 5.10E+07 | 1.21E+07 | 1.65E+06 |
| 5 month | <1.0E+05 | 4.20E+07 | 2.10E+07 | <1.0E+05 |
| 6 month | <1.0E+05 | 4.35E+07 | 2.60E+07 | <1.0E+05 |

All results reported as CFU/g. The CFU/g average of the T = 0 days is the post-processing viability average or 3.12 × 10⁷ CFU/g.

For the blister and bottle without desiccant packaging, the probiotic count fell to below 10⁵ which is considered to be less than viability levels. For the 23° C. and 50% relative humidity of the samples in the blisters packaging, the probiotics were not viable after 3 months. For the bottles without desiccant, the probiotics of Formulation 1 were no longer viable after 3 months and for Formulation 2 were no longer viable after 4 months.

Probiotics in tubes and bottles with desiccant were more stable. For the 35° C. and 85% relative humidity, the probiotics were still viable at the conclusion of the test (28 days). For the real time conditions, the probiotics were still viable after 6 months.

The stability tests for Formulations 3 and 4 are shown in Tables 12-15.

TABLE 12
Stability Results for Formulation 3
at 35° C. and 85% relative humidity.

| | | Bottle with | | Bottle w/o |
|---|---|---|---|---|
| Time | Blister | Desiccant | Tube | Desiccant |
| 0 days | 4.50E+07 | 7.60E+06 | 5.50E+06 | 3.90E+07 |
| 1 day | 1.00E+08 | 6.25E+07 | 2.80E+07 | 7.55E+07 |
| 2 days | 6.10E+07 | 7.05E+07 | 2.50E+07 | 1.19E+08 |
| 3 days | 3.00E+07 | 3.15E+07 | 3.35E+07 | 9.10E+07 |
| 5 days | 1.50E+07 | 6.95E+07 | 1.10E+08 | 4.55E+07 |
| 7 days | 1.45E+07 | 3.30E+07 | 2.10E+07 | 1.64E+07 |
| 10 days | 1.55E+08 | 6.75E+07 | 8.50E+06 | 1.80E+07 |
| 14 days | 1.55E+07 | 1.85E+07 | 1.45E+07 | 1.05E+07 |
| 21 days | <1.0E+05 | 3.40E+07 | 2.90E+07 | 1.15E+06 |
| 28 days | <1.0E+05 | 3.50E+07 | 1.51E+07 | 5.00E+05 |

All results reported as CFU/g. The CFU/g average of the T = 0 days is the post-processing viability average or 2.43 × 10⁷ CFU/g.

TABLE 13
Stability Results for Formulation 3
at 23° C. and 50% relative humidity.

| | | Bottle with | | Bottle w/o |
|---|---|---|---|---|
| Time | Blister | Desiccant | Tube | Desiccant |
| 0 days | 4.50E+07 | 7.60E+06 | 5.50E+06 | 3.90E+07 |
| 1 month | 3.60E+07 | 4.55E+07 | 3.60E+07 | 6.70E+07 |
| 2 month | 6.25E+07 | 8.25E+07 | 1.89E+07 | 3.80E+07 |
| 3 month | 2.35E+06 | 4.75E+07 | 1.67E+07 | 4.65E+06 |
| 4 month | <1.0E+05 | 3.35E+07 | 7.10E+06 | 4.50E+05 |
| 5 month | <1.0E+05 | 3.75E+07 | 7.45E+06 | <1.0E+05 |
| 6 month | <1.0E+05 | 5.95E+07 | 1.85E+07 | <1.0E+05 |

All results reported as CFU/g. The CFU/g average of the T = 0 days is the post-processing viability average or 2.43 × 10⁷ CFU/g.

TABLE 14
Stability Results for Formulation 4
at 35° C. and 85% relative humidity.

| | | Bottle with | | Bottle w/o |
|---|---|---|---|---|
| Time | Blister | Desiccant | Tube | Desiccant |
| 0 days | 6.00E+07 | 3.00E+07 | 2.80E+07 | 4.50E+07 |
| 1 day | 3.10E+07 | 2.10E+07 | 4.30E+07 | 1.80E+08 |
| 2 days | 7.55E+07 | 4.30E+07 | 4.60E+07 | 1.35E+08 |
| 3 days | 3.50E+08 | 1.49E+08 | 6.00E+07 | 9.70E+07 |
| 5 day | 7.05E+08 | 4.30E+07 | 2.80E+07 | 5.50E+08 |
| 7 days | 3.35E+07 | 3.95E+07 | 5.20E+07 | 7.50E+07 |
| 10 days | 5.45E+07 | 8.15E+07 | 6.60E+07 | 6.95E+07 |
| 14 days | 2.10E+06 | 4.30E+07 | 6.30E+07 | 3.25E+07 |
| 21 days | 4.50E+05 | 4.70E+07 | 4.55E+07 | 1.14E+07 |
| 28 days | <1E+05 | 4.25E+07 | 4.55E+07 | 1.20E+07 |

All results reported as CFU/g. The CFU/g average of the T = 0 days is the post-processing viability average or 4.075 × 10⁷ CFU/g.

TABLE 15
Stability Results for Formulation 4
at 23° C. and 50% relative humidity.

| | | Bottle with | | Bottle w/o |
|---|---|---|---|---|
| Time | Blister | Desiccant | Tube | Desiccant |
| 0 days | 6.00E+07 | 3.00E+07 | 2.80E+07 | 4.50E+07 |
| 1 month | 8.25E+07 | 2.05E+08 | 5.00E+07 | 9.00E+07 |
| 2 month | 5.55E+07 | 2.85E+08 | 1.03E+08 | 5.90E+07 |
| 3 month | 2.85E+07 | 2.15E+08 | 4.30E+07 | 4.90E+07 |
| 4 month | 9.00E+06 | 5.15E+07 | 2.15E+07 | 3.45E+07 |
| 5 month | 5.55E+06 | 7.50E+05 | 3.70E+07 | 6.25E+06 |
| 6 month | 1.40E+07 | 4.35E+07 | 4.65E+07 | 1.05E+07 |

All results reported as CFU/g. The CFU/g average of the T = 0 days is the post-processing viability average or 4.075 × 10⁷ CFU/g.

For the accelerated testing, the probiotics in the blister pack were not viable after 14 days at these conditions. For the bottles without desiccants, the probiotics were not viable after 21 days of testing. For both packaging types, the probiotics were not viable after 3 months at real time conditions. For the tubes and the bottle with desiccant, the probiotics were viable throughout the entire accelerated test of 28 days. They were also viable throughout the real time testing of 6 months. Formulation 4 had the best stability results of the four formulations.

For the accelerated testing, the bottle without desiccant, the bottle with desiccant, and the tube all passed the accelerated test (viable after 28 days). The blister pack was not as stable. For this packaging, the probiotics were not viable after 14 days. However, all packaging types passed the real time testing. All products were still viable after 6 months at 23° C. and 50% relative humidly.

In the second trial, isomalt was used as a base as it was believed that probiotics were more stable in isomalt than sorbitol due to lower hygroscopicity of isomalt. In the second trial, an isomalt based compressible base was used for two formulations (Formulations 5 and 6). As shown in Table 16, Formulations 5 and 6 were the same as Formulations 1 and 3 except for the isomalt compressible gum base.

TABLE 16
Formulations Produced in the Second Trial

| | Formulation 5 | Formulation 6 |
|---|---|---|
| Base | All In One Gum SF - | All in One Gum SF - |
| | Isomalt | Isomalt |
| Probiotics | Probiotic Preblend | Probiotic Preblend |
| | with xylitol | with xylitol |
| Flavor | Liquid Flavor at 1% | Liquid Flavor at 1.7% |
| | by weight | by weight |

The machinery and the basic procedure used in the second trial were similar to the first trial; however, the mixing time was shortened to determine its effect.

50 kg batches of master blend of each of the two formulations were prepared as well as the probiotic pre-blend. For the pre-blend, a low shear tumbling mixer (L.B. Bohle D-59320, Type LM40) was used in a humidity controlled environment. A 90:10 weight ratio of xylitol to lactobacillus plantarum 299V (Probi AB, Lund, Sweden) was used in the pre-blend and the pre-blend was mixed for 20 minutes at 30 rpm.

The master blend was weighed and added to the mixer along with the probiotic pre-blend and color. Mixing was done for 10 minutes after which the blend appeared uniform. The magnesium stearate was then added and mixed for 3 minutes.

An objective of this trial was to determine if probiotic loss observed during processing were due to the probiotics sticking to the walls of the machinery. Powder from inside the mixer was collected and analyzed. After analysis it was determined that there was not an excessive amount of the probiotics stuck to the walls of the mixer but rather probiotic loss was caused by longer mixing times. The shortened mixing time improved the viability of the probiotics.

Each formulation should have had a probiotic count of about 5.5×10⁸ CFU/g. After 10 minutes of mixing, the count for Formula 5 averaged 5.175×10⁷ or 9.4% viable and for Formula 6 averaged 1.53×10⁸ CFU/g or 29% viable. Both Formula 5 and 6 viable % are improved over Formulations 1-4 due in part because of the reduced mixing times.

Single layer tablets (1.3 g and 1.5 g) were produced from Formulations 5 and 6. All formulas were packaged in four packaging types: PVDC blisters, bottles with a desiccant added thereto, a tube that has a built-in desiccant in the cap and a flowpack with multiple pellets. The bottles and tubes were manually packed and did not contain desiccant. Tubes with attached desiccant were used. The blisters and metalized flowpacks were packed on an automated line. All four package types were put into stability testing at both accelerated and real time conditions.

The formulations and packaging were tested at two conditions: 35° C. and 85% relative humidity (for accelerated shelf life testing) and 23° C. and 50% relative humidity. Tables 17-20 show the bacteria counts for the different test conditions for Formulations 5 and 6.

TABLE 17
Stability Results for Formulation 5
at 35° C. and 85% relative humidity.

| | | Bottle with | | |
|---|---|---|---|---|
| Time | Blister | Desiccant | Tube | Flowpack |
| 0 days | 1.25E+07 | 6.65E+07 | 2.30E+07 | 1.05E+08 |
| 3 days | 6.55E+07 | 1.08E+08 | 3.35E+07 | 1.17E+08 |
| 7 days | 8.00E+07 | 7.10E+07 | 4.00E+07 | 9.90E+07 |
| 10 days | 1.35E+07 | 6.80E+07 | 2.10E+07 | 3.90E+07 |
| 14 days | 5.00E+07 | 2.10E+07 | 3.75E+07 | 5.25E+07 |
| 21 days | 1.50E+07 | 3.20E+07 | 2.70E+07 | 4.10E+07 |
| 28 days | 8.60E+06 | 1.55E+07 | 5.95E+07 | 1.75E+07 |

All results reported as CFU/g. The CFU/g average of the T = 0 days is the post-processing viability average or 5.175 × 10⁷ CFU/g.

TABLE 18
Stability Results for Formulation 5
at 23° C. and 50% relative humidity.

| | | Bottle with | | |
|---|---|---|---|---|
| Time | Blister | Desiccant | Tube | Flowpack |
| 0 days | 1.25E+07 | 6.65E+07 | 2.30E+07 | 1.05E+08 |
| 1 month | 4.00E+07 | 3.25E+07 | 2.80E+07 | 5.90E+07 |
| 2 month | 8.50E+07 | 6.98E+07 | 4.95E+07 | 7.00E+07 |
| 3 month | 3.35E+07 | 8.00E+07 | 3.15E+07 | 5.10E+07 |
| 4 month | 2.00E+07 | 1.42E+08 | 2.95E+07 | 3.45E+07 |
| 5 month | 2.85E+06 | 5.40E+07 | 4.15E+07 | 9.55E+06 |

All results reported as CFU/g. The CFU/g average of the T = 0 days is the post-processing viability average or 5.175 × 10⁷ CFU/g.

TABLE 19
Stability Results for Formulation 6
at 35° C. and 85% relative humidity.

| | | Bottle with | | |
|---|---|---|---|---|
| Time | Blister | Desiccant | Tube | Flowpack |
| 0 days | 2.55E+08 | 1.06E+08 | 1.75E+07 | 1.80E+08 |
| 3 days | 1.55E+08 | 8.40E+07 | 3.70E+07 | 1.06E+08 |
| 7 days | 8.85E+07 | 1.60E+08 | 3.60E+07 | 1.07E+08 |
| 10 days | 2.95E+07 | 1.66E+08 | 4.65E+07 | 4.20E+07 |
| 14 days | 3.40E+07 | 7.65E+07 | 3.40E+07 | 3.70E+07 |
| 21 days | 1.29E+07 | 3.40E+07 | 1.50E+07 | 3.25E+07 |
| 28 days | 8.90E+06 | 1.50E+07 | 3.40E+07 | 3.65E+07 |

All results reported as CFU/g. The CFU/g average of the T = 0 days is the post-processing viability average or 1.53 × 10⁸ CFU/g.

TABLE 20
Stability Results for Formulation 6
at 23° C. and 50% relative humidity.

| | | Bottle with | | |
|---|---|---|---|---|
| Time | Blister | Desiccant | Tube | Flowpack |
| 0 days | 2.55E+08 | 1.06E+08 | 1.75E+07 | 1.80E+08 |
| 1 month | 6.85E+07 | 6.25E+07 | 6.35E+07 | 1.28E+08 |
| 2 month | 6.75E+06 | 9.80E+07 | 4.65E+07 | 2.47E+07 |
| 2 month | 5.30E+07 | Due to the low blister bug count, the 2 | | |
| | | month blister was retested (avg of 4) | | |
| 3 month | 3.65E+07 | 6.90E+07 | 4.15E+07 | 3.70E+07 |
| 4 month | 2.40E+06 | 1.60E+07 | 2.70E+07 | 3.25E+07 |
| 5 month | | | 2.55E+07 | 1.40E+07 |

All results reported as CFU/g. The CFU/g average of the T = 0 days is the post-processing viability average or 1.53 × 10⁸ CFU/g.

For Formulation 5, all packaging types passed both accelerated (28 days) and real time testing (5 months). For the blister and flowpack, 1 order of bacteria was lost over the entirety of the test for both the accelerated and real time conditions. The tube and bottle with desiccant remained stable throughout the testing for both conditions.

For Formulation 6, all packaging types passed the accelerated testing. Over the entire accelerated testing (28 days), the blister package lost 2 orders of magnitude of probiotics. The bottle with desiccant and the flow pack lost 1 order over the testing period. The tube packaging was the most stable for Formulation 6. Over the course of the test, it did not loss any viability compared to the initial bacteria count.

The blisters and bottles with desiccant for Formulation 6 were tested for 4 months under real time conditions. The tubes and flowpacks completed 5 months of real time testing. All the packaging types passed the real time testing, whether it was 4 months or 5 months. The results for the real time testing trended the same as the accelerated results. The blister pack lost 2 orders of magnitude and the bottles with desiccant lost one order over the 4 month test. The flowpack also lost 1 order over the 5 month testing period. Again, the tube is the most stable packaging form. No bacteria were lost over the 5 month test period for the tube packaging.

Formulations 5 and 6 were generally more stable than Formulations 1-4. It is believed the lack of stability is attributable to the hygroscopicity of sorbitol and the effect of moisture uptake on the probiotics. Sorbitol is a highly hygroscopic polyol whereas xylitol, maltitol, mannitol, isomalt and erythritol have low degrees of hygroscopicity. The bacteria in Formulations 5 and 6 survived longer in the blister packages for both the accelerated and real time testing. The other packaging types that the two trials have in common, bottle w/ desiccant and tubes, performed similarly under both conditions.

### Example 4

### Preparation of a Two Layered Compressed Chewing Gum

This Example describes preparation of the two layered gum. While the two-layered gum of this example contains probiotics, it should be understood that similar gums that do not contain probiotics could be produced as described.

Two batches (400 kg each) of Formulation 7 shown in Table 18 were produced as described more fully below.

TABLE 21
Formulation Produced in Example 4

| | Formulation 7 |
|---|---|
| Base | All In One Gum Isomalt with additional |
| | Sorbitol |
| Probiotics | No preblend |
| Flavor | Liquid Flavor at 1.4% by weight; |
| | Spray Dried Flavor at 0.6% by weight; |
| | Spray Dried Menthol at 0.3% by weight. |

The batches (400 kg each) were produced in a large “V” blender. All ingredients were weighed in a humidity controlled room. The base was weighed and mixed with sorbitol, high intensity sweetener HIS, spray dried menthol, and spray dried flavor. The talc and base were added to the mixer and mixed for 4 minutes. The flavor was added over 18 minutes while mixing. After the flavor was added, the ingredients were mixed for another 7 minutes. Probiotics (lactobacillus plantarum 299V) and coloring were then added to the mixer and mixing continued for another 5 minutes. Magnesium stearate was added and mixing continued for 3 minutes.

After overnight storage, the mixture was then pressed into a two layered tablet. Both 1.3 g and 1.5 g weight samples were pressed (Fette 3090 Bi-layered (Tp9B2 and TP9B1)).

Pressing trials performed at a later time were performed to determine how to improve viability of probiotic and to develop a two layered gum with layers that sufficiently adhered to each other. In the pressing trials, the settings shown in Table 22 were utilized.

TABLE 22
Compression Settings Utilized to Form Two-Layered Gum

| Setting | Layer 1 | Layer 2 |
|---|---|---|
| Tablets/h (×1000) | 100 | 100 |
| Rotor Speed (1/min) | 34 | 34 |
| Fillomatic Speed (1/min) | 30 | 35 |
| Allowed Punch Load (kN) | 98 | 98 |
| Pre compression force (kN) | 0 | 0 |
| Main Pressure Force (kN) | 4 | 21 |
| Filling Depth (mm) | 6.3 | 10.1 |
| Pre compression punch | 4 | 2.6 |
| penetration (mm) | | |
| Main compression punch | 5.5 | 2.5 |
| penetration (mm) | | |

The tablets produced using the settings of Table 22 contained layers that adhered well together.

The formulations were packaged in 6 packaging types: PVDC blisters, bottles with a desiccant added, a tube with a built-in desiccant in the cap, bottles with a built-in desiccant in the cap, and 2 flowpacks with individual pellets. The tubes were manually packed. The blisters, bottles, and flowpacks were packed on an automated line. The stability data is shown in Tables 23 and 24.

TABLE 23
Stability Results for Formulation 7 at 35° C. and 85% relative humidity.

| | | | Bottle | | | | |
|---|---|---|---|---|---|---|---|
| | | | with | Wrigley | | Flow | Flow |
| Time | | Blister | Desiccant | Bottle | Tube | wrap #1 | wrap #2 |
| 0 | days | 9.80E+08 | 9.80E+08 | 9.80E+08 | 9.80E+08 | 3.05E+08 | 9.80E+08 |
| 3 | days | 1.39E+08 | 1.65E+08 | 4.55E+08 | 3.10E+08 | 1.26E+07 | 7.50E+07 |
| 7 | days | 2.35E+07 | 4.85E+07 | 2.65E+08 | 1.10E+08 | 4.10E+07 | 1.70E+07 |
| 10 | days | 3.75E+06 | 9.50E+06 | 1.34E+08 | 7.40E+07 | 3.45E+06 | 2.30E+06 |
| 14 | days | 2.20E+06 | 3.40E+06 | 3.55E+07 | 5.85E+07 | 6.50E+05 | 9.00E+05 |
| 21 | days | 2.50E+05 | <100000 | 2.80E+07 | 9.80E+07 | <100000 | <100000 |
| 28 | days | <100000 | <100000 | 7.10E+06 | 2.00E+07 | <100000 | <100000 |
| 5 | weeks | <100000 | <100000 | 1.65E+05 | 7.20E+06 | <100000 | <100000 |
| 6 | weeks | <100000 | <100000 | 3.50E+05 | 6.55E+06 | <100000 | <100000 |
| 7 | weeks | <100000 | <100000 | 2.50E+05 | 2.10E+06 | <100000 | <100000 |
| 8 | weeks | <100000 | <100000 | <100000 | 8.50E+05 | <100000 | <100000 |

All results reported as CFU/g.

TABLE 24
Stability Results for Formulation 7 at 23° C. and 50% relative humidity.

| | | Bottle | | | | |
|---|---|---|---|---|---|---|
| | | with | Wrigley | | Flow | Flow |
| Time | Blister | Desiccant | Bottle | Tube | wrap #1 | wrap #2 |
| 0 days | 9.80E+08 | 9.80E+08 | 9.80E+08 | 9.80E+08 | 9.80E+08 | 9.80E+08 |
| 1 month | 5.40E+07 | 1.25E+08 | 5.05E+08 | 5.60E+08 | 1.20E+08 | 6.90E+07 |
| 2 month | 2.85E+06 | 2.90E+07 | 6.05E+07 | 6.25E+07 | 2.55E+07 | 1.95E+07 |

All results reported as CFU/g.

Before compression, the actual count of probiotics in Formulation 7 was 9.033×10⁸ CFU/g. After mixing into a chewing gum powder but prior to compression, the actual count of probiotics in was measured to be 2.2×10⁸ CFU/g. After compression into dual layer tablets, the count was measured to be 9.8×10⁸ CFU/g; therefore, the viable % after the improved dual compression process is 22%.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1-54. (canceled)

55. A probiotic-containing chewing gum composition comprising
a chewing gum having a soluble and insoluble portion;
an inactive probiotic, wherein the inactive probiotic is activatable upon contact with water;
the inactive probiotic incorporated into the chewing gum under conditions which would not render the inactive probiotic ineffective prior to consumption; and
the probiotic-containing chewing gum composition has a probiotic effectiveness shelf life greater than six months.

56. A probiotic-containing chewing gum composition of claim 55 wherein the probiotic-containing chewing gum composition is a compressed chewing gum.

57. A probiotic-containing chewing gum composition of claim 55 wherein the probiotic-containing chewing gum composition is a layered chewing gum.

58. A probiotic-containing chewing gum composition of claim 55 wherein the inactive probiotic incorporated into the chewing gum is subjected to no more than 3 weight percent water.

59. A probiotic-containing chewing gum composition of claim 58 wherein the inactive probiotic incorporated into the chewing gum is subjected to no more than 1 weight percent water.

60. A probiotic-containing chewing gum composition of claim 55 in which the inactive probiotic incorporated into the chewing gum is subjected to no more than 55° C.

61. A probiotic-containing chewing gum composition of claim 55 in which the inactive probiotic incorporated into the chewing gum is subjected to a temperature above 55° C. and rapidly cooled to a temperature below 55° C.

62. A probiotic-containing chewing gum composition of claim 55 in which the inactive probiotic incorporated into the chewing gum is subjected to a temperature between 40° C. and 50° C.

63. A probiotic-containing chewing gum composition of claim 55 in which the probiotic-containing chewing gum includes a coating and at least a portion of the inactive probiotic is contained in the coating.

64. A probiotic-containing chewing gum composition of claim 55 in which the probiotic-containing chewing gum includes a center filling wherein at least a portion of the inactive probiotic is contained in the center filling.

65. A probiotic-containing chewing gum composition of claim 55 in which the inactive probiotic is selected from the group consisting of Lactobacillus acidophilus, Lactobacillus reuteri, Lactobacillus plantarum, Lactobacillus salvalarius, Lactobacillus sporogenes, Bifidobaterium longum and mixtures thereof.

66. A method of forming a probiotic-containing chewing gum comprising
incorporating at least one inactive probiotic into a chewing gum under conditions which would not render the inactive probiotic ineffective prior to consumption and would allow the inactive probiotic to be activatable upon consumption; and
maintaining the probiotic-containing chewing gum, during forming, under conditions such that the probiotic-containing chewing gum has a probiotic effectiveness shelf life greater than six months.

67. A method of claim 66 in which the forming of the probiotic-containing chewing gum includes a step of compressing to form a tablet.

68. A method of claim 66 in which the forming of the probiotic-containing chewing gum includes a step of adding a center filling wherein at least a portion of the inactive probiotic is incorporated into the center filling.

69. A method of claim 66 in which the forming of the probiotic-containing chewing gum includes a step of adding a coating wherein at least a portion of the inactive probiotic is incorporated into the coating.

70. A method of claim 66 in which the inactive probiotic is incorporated into the chewing gum and is subject to no more than 1 weight percent water.

71. A method of claim 66 in which the forming of the probiotic-containing chewing gum includes the steps of preblending the inactive probiotic with a bulking agent to form a pre-blend and mixing the pre-blend with a chewing gum base.

72. A method of claim 66 in which a moisture resistant pre-coating is applied to the probiotic-containing chewing gum and the probiotic-containing chewing gum is coated using an aqueous carrier.

73. A method of claim 72 in which the moisture resistant pre-coating is selected from the group consisting of gum talha, magnesium stearate, silicon dioxide, titanium dioxide, and aluminium oxide.

74. A method of claim 66 in which at least a portion of the inactive probiotics are applied to the surface of the chewing gum.

75. A method of claim 66 in which the probiotic-containing chewing gum composition is formed into a layered chewing gum, containing at least two layers, wherein a first layer is formed by compression at less than 10 kN and a second chewing gum layer is formed by compression at less than 30 kN, the inactive probiotic being incorporated into at least one of the layers.

76. A method of claim 66 in which the inactive probiotic is selected from the group consisting of Lactobacillus acidophilus, Lactobacillus reuteri, Lactobacillus plantarum, Lactobacillus salvalarius, Lactobacillus sporogenes, Bifidobaterium longum and mixtures thereof.
